(19) United States
(12) Patent Application Publication
(10) Pub. No.: US 2024/0050254 A1
(43) Pub. Date: Feb. 15, 2024

(54) DEVICES AND METHOD FOR BI-DIRECTIONAL ADJUSTMENT OF INTRAGASTRIC BALLOONS, AND SHUT-OFF VALVE

(71) Applicant: Leonardo SALLES DE ALMEIDA, MG, 30180-ll l Belo Horizonte (BR)

(72) Inventor: Leonardo SALLES DE ALMEIDA, MG, 30180-ll l Belo Horizonte (BR)

(21) Appl. No.: 17/641,219

(22) PCT Filed: Sep. 21, 2020
(86) PCT No.: PCT/BR2020/050374

§ 371 Date: Mar. 8, 2022

(30) Foreign Application Priority Data
Sep. 20, 2019 (BR) 10 2019 019657 2
Sep. 1, 2020 (BR) 10 2020 017 878 4

(51) Int. Cl. A61F 5/00; A61M 39/12; A61M 39/22; A61M 99/00; A61M 39/26

(52) U.S. Cl. CPC A61F 5/0036; A61F 5/0089; A61F 5/003; A61M 39/12; A61M 39/223; A61M 99/00; A61M 39/26; A61M 2039/1077

(57) ABSTRACT

The present devices and method are related to treatments for obesity and for bi-directional adjustment (inflation/deflation) of intragastric balloons, thus allowing the inflation or deflation of the volume of the intragastric balloon already in a patient's stomach. A first device is configured to be introduced into the valve of the intragastric balloon and to be retained therein, and comprising a coupling element coupled to the body of the first device and configured to project outwards from the valve of the intragastric balloon. A second device comprising a tubular extension is configured to pass through the valve of the intragastric balloon, thus neutralizing its valve effect and allowing the bi-directional adjustment of same. A method is disclosed that carries out the steps of positioning an intragastric balloon already in the stomach using the first device. A shut-off valve configuration is disclosed for use with an implantable catheter.

### TECHNICAL DOMAIN

The present invention relates to the field of treatments for obesity, more specifically, to the provision of devices and a method for the bi-directional adjustment (inflation/deflation) of intragastric balloons, thus allowing the inflation or deflation of the volume of the intragastric balloon already in the patient's stomach.

### BASICS OF THE INVENTION

Various obesity treatments are widely known in the prior art. The use of intragastric balloons as a weight-loss treatment began in the mid-1980s in the United States of America. They were introduced as an alternative preliminary intervention before gastric surgery.

The first balloons were cylindrical in shape, waterproof, and had a simple self-sealing valve. The balloons were endoscopically inserted into the patient's stomach and then inflated with air. The intragastric balloon then remained loose in the patient's stomach and was designed to be removed after a period of approximately 4 months.

However, as the use of these first balloons increased and they began to be used extensively worldwide, some problems began to manifest themselves. Patients' weight loss was temporary as it gradually returned, and in some cases the balloons deflated spontaneously or obstructed the passage of food through the stomach. They also caused gastric erosions and ulcers in patients. Furthermore, in some cases, during the 4-to-6-month implantation period, the intragastric balloon (foreign body) can become a gastric irritant, causing nausea and/or discomfort, leading to early removal.

Due to these complications, the use of intragastric balloons was frowned upon by the United States Food and Drug Administration (FDA) and its use was consequently and abruptly discontinued in the United States. It was not until 2015 that the FDA approved the use of another type of intragastric balloon in the United States.

In 1987, during a multidisciplinary conference in Florida with international experts from the fields of gastroenterology, surgery, obesity, nutrition, and behavioral medicine, some recommendations were defined to improve the safety and efficacy of future intragastric balloons. The conference defined the following recommendations for intragastric balloons:
1) they are efficient in promoting weight loss;
2) they are filled with liquid instead of air;
3) be able to be adjustable;
4) have a smooth surface with low potential to cause ulcers and obstructions;
5) contain a radiopaque marker; and
6) be made of durable materials.

Despite the lack of approval of intragastric balloons for treatment in the United States, the use and advances in intragastric balloon technologies continued to be pursued in several countries such as Australia, Canada, Mexico, India, Guatemala, and countries in Europe and South America.

During the next decades, more efficient intragastric balloons were developed following the recommendations defined at the 1987 conference. New balloons were developed to allow insertion, adjustment, and subsequent removal of intragastric balloons with minimal aggression to the patient's organs.

The balloons available today are generally spherical, made of medical grade silicone elastomeric material, and are usually filled with an aqueous solution. This aqueous solution is usually a saline solution.

Balloon placement is performed without the need for complex surgical procedures. The intragastric balloon placement process usually takes approximately 20 to 30 minutes.

The process is performed via endoscopy using, where appropriate, local anesthesia and sedation. Placement is temporary and intragastric balloons are usually removed after six to twelve months.

The acceptance of an intragastric balloon and the effectiveness of treatment through its use is directly linked to the volume displaced in the stomach by it. Therefore, the possibility of adjusting an intragastric balloon is beneficial, as it prevents early removals due to balloon intolerance.

In some cases, the patient may not tolerate the presence of the balloon, causing nausea and uncontrollable vomiting. In other cases, the patient may have a decreased or even stopped feeling of satiety due to the balloon. In these cases, we can increase the volume of the balloon and restore prolonged satiety even with little food intake.

With the bi-directional adjustment of an intragastric balloon it is possible to decrease or increase the balloon volume, allowing differentiated implantation strategies. Such differentiated strategies are necessary in nephropathic patients, elderly patients, patients with panic syndrome, and other special cases. In these cases, it is preferred to start with a small volume balloon and then gradually increase the volume. On the other hand, the adjustment allows the volume of the intragastric balloon to be increased during treatment in cases of low weight loss, thus optimizing the effects of the intragastric balloon.

However, among the intragastric balloon technologies in use today, most do not allow for adjustability (inflate/deflate), making it difficult for patients in general to adapt. The most popular types of intragastric balloons available today are the Orbera® model manufactured by Apollo and the Corporea model manufactured by Medicone.

One adjustable balloon model available on the market is the endogAst®, an Adjustable Totally Implantable Intragastric Prosthesis (ATIIP). This balloon model involves a mini-invasive technique for the treatment of morbid obesity. The endogAst® implantation is performed through a surgical and endoscopic procedure. After the endoscopic procedure for balloon implantation, a subcutaneous system is attached to the abdominal wall, connected to the air-inflated prosthesis in the gastric body. It is evident that such an adjustable balloon model necessarily requires the additional complication of subjecting a patient to invasive surgery, resulting in increased risks of infection to the patient during implantation. Also, a longer recovery time is required post-surgery.

It should be noted that if the patient rejects the implantation of this intragastric balloon, a complex invasive surgery and endoscopy procedure would be required to remove this system.

Another adjustable balloon model available on the market is the Spatz3. However, it has a coarse adjustment stem that often causes ulcers. These ulcers occur due to the pressure that the semirigid stem causes on the gastric wall. The ulcers occur mostly in the angular incisure of the stomach, but they can occur on the body and even the gastric fundus of the stomach. Sometimes, in more severe cases, these ulcers can deepen, leading to gastric perforation, with sepsis and a risk of death for the patient.

### PRIOR ART

Some examples of intragastric balloons from the prior art will be discussed below. More specifically, the execution of the valves and their means of adjusting will be discussed.

An example of intragastric balloon valves and adjustment means is described in patent application PI 1001214-1. The document discloses two kits, one for balloon insertion and the other for balloon removal and adjustment. The balloon contains an hourglass-shaped shut off valve and is to be passed through a tubular inflation needle. The shut off valve is attached to a nipple that is internally threaded.

The balloon valve of PI 1001214-1, depending on the configuration, is provided with a nipple, magnet, or coupling element. In configurations that have the coupling element, a pull wire or an endoscopic clamp can be used to hold the balloon. It is a preferred configuration of PI 1001214-1 to have a proximal end (which is externalized for balloon inflation) with a thread.

As would become apparent to an expert on the subject, the cited document reveals an intragastric balloon that contains a series of apparently rigid protrusions on the balloon valve, which would invariably cause ulcers on the gastric wall by the normal displacement of the balloon.

Still referring to the balloon valve, it carries a grip tab, made of polymeric material, which suggests that it has some considerable rigidity and therefore would also be likely to cause ulcers in the gastric wall.

Additionally, the paper reveals a method of attachment involving magnets, threads, and tabs that results in a high degree of complexity during intragastric balloon adjustment.

Another example of intragastric balloon valves and adjustment means is described in patent application US 2001037127. The document discloses a slit valve for use with an inflatable medical device having a flange surface with an opening in it. The slit valve has a valve body connected to the flange surface and a chamber formed in the valve body to accept a supply pipe inserted through the opening in the flange surface.

The slit valve has a concave section at one or both ends, which are connected by a slit formed in the valve body.

Also revealed is a filler tube that has its insertion tip inserted into the flange opening of the slitted bi-directional valve.

Additionally, the document discloses the possibility of inserting a small diameter probe through the valve slit, thus allowing the intragastric balloon to be deflated. Furthermore, it is mentioned that the valve provides a device that does not leak under normal conditions but allows the passage of bi-directional fluid under required conditions.

The above document, however, reveals the intragastric balloon valve and a filling tube that allows only for balloon inflation. Furthermore, the document merely mentions the possibility of introducing a small diameter probe for balloon deflation. The bi-directional adjustment for inflation/deflation while the balloon remains in the patient's stomach is not addressed by US 2001037127.

Additional examples of intragastric balloon valves and means of adjustment are described in applications US 20150230956 and US 2015230957.

Document US 20150230956 discloses an intragastric balloon system that claims an intragastric balloon comprising a valve used in conjunction with the filling tube in an intragastric balloon.

However, the filler tube revealed by application US 20150230956 is calibrous and cannot be implanted in the patient, and is designed to be removed during balloon implantation, leaving no possibility for future adjustments to that balloon.

The document US 2015230957, on the other hand, discloses a method of preparing an intragastric balloon for inflation after it has been implanted in a patient. The preparation of the intragastric balloon follows steps like those disclosed in the system of application US 20150230956, and then the prepared balloon is inserted into a patient for inflation. Like document US 20150230956, the present document discloses that the filling tube is designed to be removed from the intragastric balloon after it is filled, leaving no possibility for future adjustments to that balloon.

Another document, US2003158569, reveals a device for the treatment of morbid obesity. The implantation is performed through a surgical and endoscopic procedure. To fit the balloon, a subcutaneous system must be attached to the abdominal wall, resulting in the additional complication of subjecting a patient to invasive surgery, resulting in a higher risk of infection to the patient during implantation, and a longer recovery time after surgery. Also, in case of patient intolerance, a new complex invasive surgery and endoscopy procedure would be required for the removal of this device.

Seeking to address the deficiencies of the prior art, the present invention provides devices and a method for the bi-directional adjustment of intragastric balloons, enabling adjustment by inflation/deflation of the intragastric balloon while in the patient's stomach with reduced incidence of ulcers.

Furthermore, the practicality of having the devices implemented for bi-directional adjustment by a physician would reduce the sedation and/or anesthesia time for the patient and the risk of trauma to the patient's esophagus and stomach.

Further, the devices of the present invention enable the use of lower cost inflatable/deflatable balloons than conventional limited fit balloons while retaining the benefits of balloons with higher cost inflation/deflation characteristics.

### SHORT SUMMARY OF THE INVENTION

The present invention falls within the field of treatments for obesity, more specifically, in the provision of devices and a method for the bi-directional adjustment (inflation/deflation) of intragastric balloons, thus allowing the inflation or deflation of the intragastric balloon volume already in the patient's stomach. Accordingly, the present invention presents a first device configured to be introduced into the intragastric balloon valve and to be retained therein and comprising a coupling element, coupled to the body of the first device and configured to project out of the intragastric balloon valve.

In addition, a second device is presented that comprises a tubular extension configured to pass through the valve of the intragastric balloon, thus neutralizing its valve effect and allowing its bi-directional adjustment.

Also shown is an intragastric balloon assembly, containing one of the bi-directional adjustment devices and an implantable catheter claimed herein, already integrated into its valve.

Additionally, a method that performs the positioning steps of an intragastric balloon already in the stomach with the use of the said first device is presented. In view of the above, the present invention provides devices and a method for the bi-directional adjustment of intragastric balloons, enabling adjustment by inflation/deflation of the intragastric balloon while in the patient's stomach with reduced incidence of ulcers.

Finally, a shut off valve is presented, adapted to be attached to an implantable catheter of an intragastric balloon, thus preventing its deflation while in a patient's stomach.

### SHORT SUMMARY OF THE DRAWINGS

The figures referred to are shown, according to a preferred configuration of the present invention:

FIG. 1 is a front view of a first device 100 for bi-directional adjustment with a coupling element 120.

FIG. 2 is a top perspective view of a first device 100 for bi-directional adjustment with a coupling element 120.

FIG. 3 is a bottom perspective view of a first device 100 for bi-directional adjustment with a coupling element 120.

FIG. 4 is a top view of a first device 100 for bi-directional adjustment with a coupling element 120.

FIG. 5 is a side view of a second device 200 with a tubular extension portion 204 for bi-directional adjustment.

FIG. 6 is a sectional view of a second device 200 with a tubular extension portion 204 for bi-directional adjustment.

FIG. 7 is a top view of a second device 200 with a tubular extension portion 204 for bi-directional adjustment.

FIG. 8 is a front view with magnification of a mandrel 400 inserted into an implantable catheter 300.

FIG. 9 is a side view with magnification of a second device 200 with a tubular extension portion 204 for bi-directional adjustment coupled to an implantable catheter 300 by a mandrel 400.

FIG. 10 is a front view with magnification of a second device 200 with a tubular extension portion 204 for bi-directional adjustment coupled to an implantable catheter 300 by a mandrel 400.

FIG. 11 is a view of a device 500 with a catheter 501 and a connector 503 at the proximal end and a tube 502 at the distal end.

FIG. 12 is a sectional perspective view of a valve of an intragastric balloon 600 with the first device 100 attached to it.

FIG. 13 is a front cross-sectional view of a valve of an intragastric balloon 600 with the first device 100 attached to it.

FIG. 14 is a cross-sectional side view of a 600 intragastric balloon valve with the first device 100 attached to it.

FIG. 15 is an illustrative view of an assembly 700 containing an intragastric balloon 701, manufactured with a bi-directional adjustment device 702 and an implantable catheter 703 integral to it.

FIG. 16 is an illustrative perspective view of the components of a shut off valve 800.

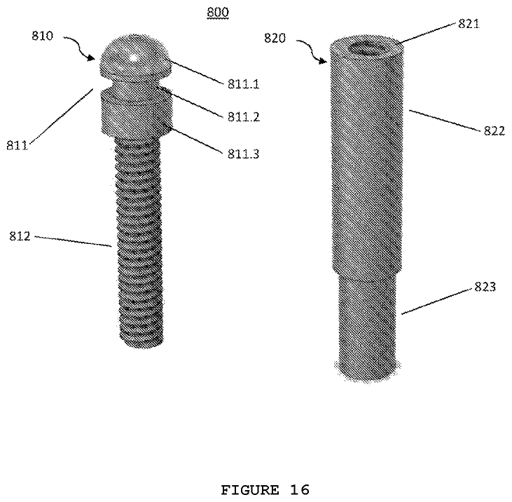

FIG. 17 is a sectional view of the components of a shut off valve 800.

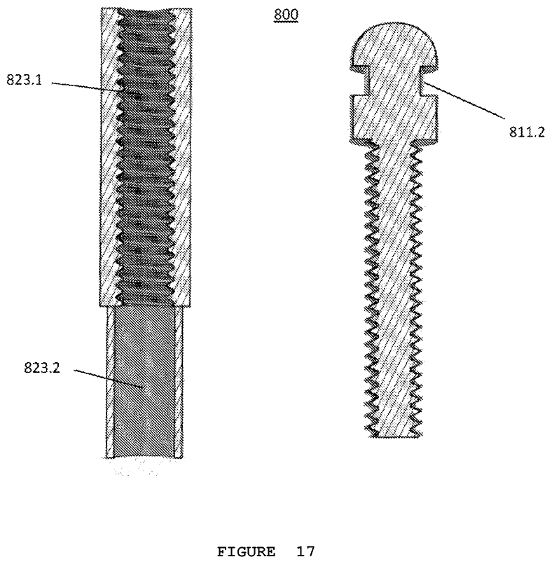

### DETAILED DESCRIPTION OF THE INVENTION

As previously described, the present invention relates to devices 100; 200 for the bi-directional adjustment (inflation/deflation) of intragastric balloons. Further, methods for the bi-directional adjustment of intragastric balloons and a shut off valve are described.

In general, the present invention is designed to be coupled to an intragastric balloon valve, such as a slitted intragastric balloon valve.

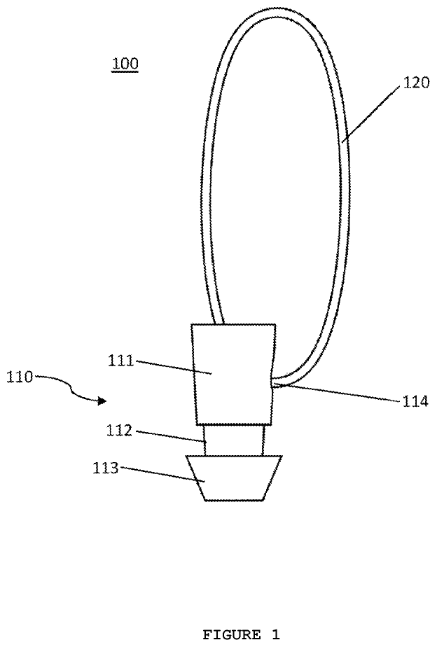

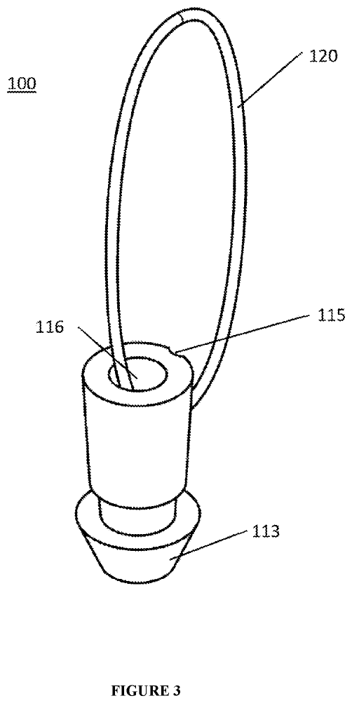

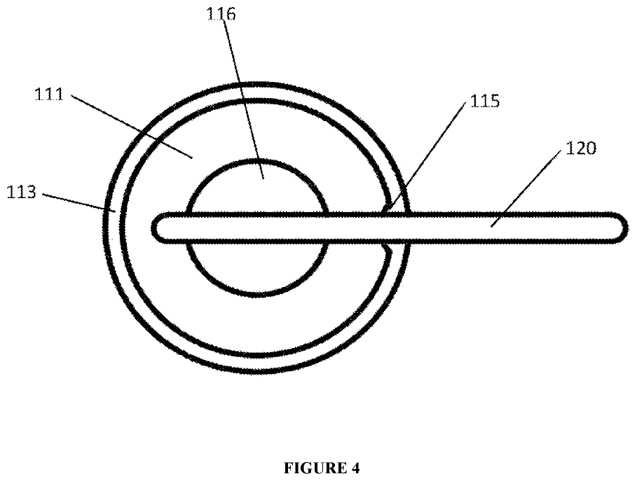

According to a first realization of the present invention, exemplified by FIGS. 1, 2, 3 and 4, a first device 100 for bi-directional adjustment of an intragastric balloon is provided.

The first device 100 for bi-directional adjustment of an intragastric balloon comprises a body 110 and a coupling element 120. The body 100 further comprises an upper portion 111, a neck portion 112, an anchor portion 113 and a central cavity 116. The upper body portion 111 further comprises a lateral hole 114 and a lateral recess 115 in the upper body portion 111.

The body 110 of the first device 100 is set to be introduced, until the body 110 is completely introduced into the intragastric balloon valve, to be retained in it by means of the anchor portion 113.

The body 110 of the first device 100 is preferably manufactured of a biocompatible polymeric material.

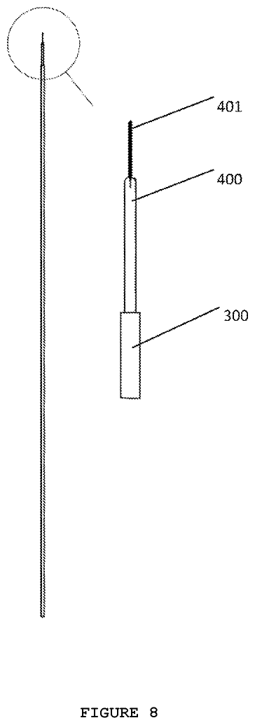

For the introduction of the first device 100 into the intragastric balloon valve, a shorter version of the mandrel 400 of only about 5 cm is used, as exemplified by FIG. 8, which is preferably manufactured of a metallic material. The mandrel 400 is intended to be inserted into the central chamber 116 of the body 110 to provide rigidity to the assembly to then enable insertion of the first device 100 into the intragastric balloon valve. After insertion of the first device 100 into the intragastric balloon valve, the mandrel 400 is removed from the device 100 so that the balloon, already with the bi-directional adjustment device 100, can be implanted in a patient.

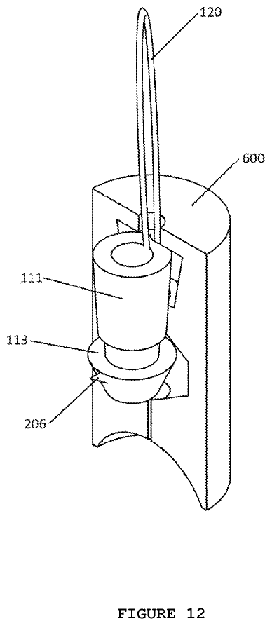

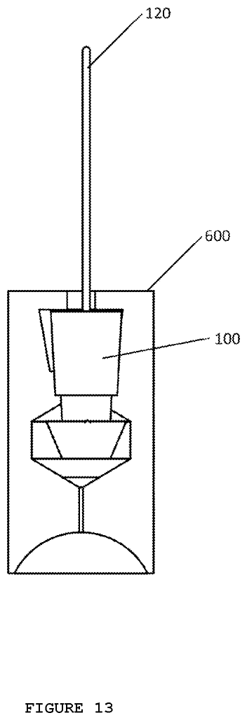

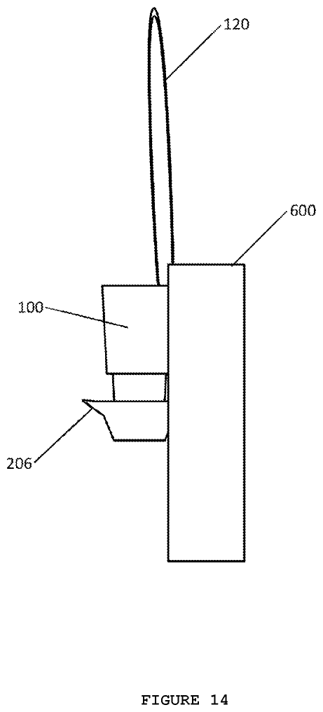

The anchor portion 113 has a tapered shape, designed to pass through the distal end of the intragastric balloon valve until the entire anchor portion 110 is inserted into the valve, causing the first device 100 to be retained in the intragastric balloon valve, as exemplified by FIGS. 12, 13 and 14.

The neck portion 112 of the first device 100, as exemplified in FIGS. 1, 2 and 3, has the function of providing a fluid seal when in contact with the walls of the intragastric balloon valve. Further, the neck portion 112 assists in securely coupling the anchor portion 110.

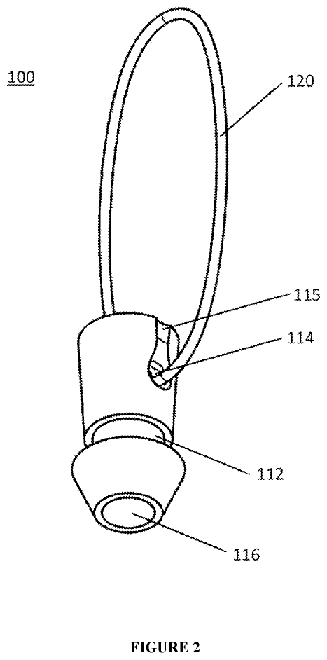

The upper portion 111 of the body 110 of the first device 100 for bi-directional adjustment of an intragastric balloon further comprises a lateral recess 115 and a lateral hole 114, as exemplified in FIG. 2.

The lateral hole 114 provides engagement of the coupling element 120 with the upper portion 111 of the body 110 of the first device. The lateral hole 114 passes through the side of the first device 100 until it reaches the central cavity 116 thereof, allowing the coupling element 120 to sit in the hole 114.

Further, a lateral recess 115 is provided, adjacent the lateral hole 114, to allow the coupling element 120 to be removably engaged therein when the coupling element 120 is pulled. The coupling element 120 is manufactured of a polymeric material, preferably Prolene.

The central cavity 116 of the first device may be smooth. Additionally, the central cavity 116 is configured to be coupled by means of a smooth fit to a device 500 introduced by an endoscopy apparatus.

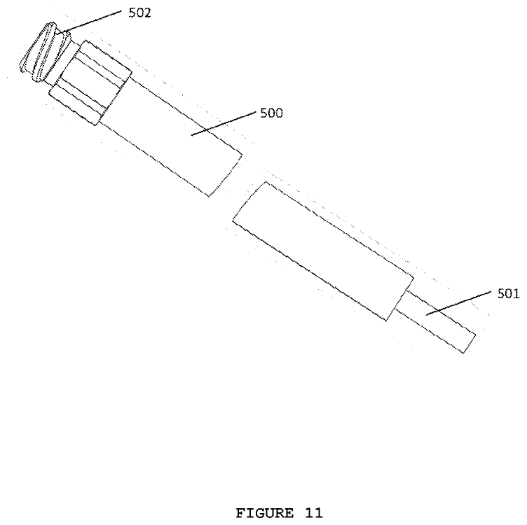

The device 500 is used in conjunction with the first device 100 to perform the balloon adjustment in the patient. The device 500 consists of a catheter 501, a connector 503 at its proximal end and a tube 502 at its distal end. The catheter 501 is preferably made of PTFE (Polytetrafluoroethylene) and the connector 503 is preferably of the Luer Lock type, as exemplified in FIG. 11. Further, the tube 502 at the distal end of the device 500 is preferably manufactured of a metallic material.

During the adjustment, the coupling element 120 is used, which is grasped by an endoscopic forceps inserted together with the device 500, to grasp the first device 100, attached to the intragastric balloon, and allow the tube 502 to penetrate the balloon valve, promoting its adjustment.

Additionally, the present invention comprises a method for performing intragastric balloon adjustment with the use of the former device 100 already in the patient's stomach by means of an endoscope.

This method aims to provide a new and improved way of fitting an intragastric balloon that minimizes the potential for causing ulcers and obstructions in the patient.

That is, normally the valve of an intragastric balloon already in the patient's stomach, due to gravity and other displacements resulting from patient movement, will be positioned distally to the stomach inlet. Therefore, when locating the balloon with the endoscope by visual means, which can be an image or digital video, it is necessary to pass the endoscope through the side of the balloon to gain access to the adjustment element 120 attached to the balloon valve. However, due to the friction between the endoscope and the balloon, the balloon rotates together with the endoscope, i.e. in the opposite direction to the patient's esophagus. Such movement prevents access to the coupling element 120 attached to the balloon valve and consequently balloon adjustment.

Said method comprises the following steps:
a) placing a silicone sleeve over the endoscopy tube before introduction into the patient;
b) Insert the endoscopy tube with the silicone sleeve orally into the patient until the stomach is reached;
c) Upon reaching the stomach, locate the balloon with the endoscope by visual means;
d) When you visually identify that the balloon valve is in a position that prevents access to the 120 coupling element, pass the endoscope through the side of the balloon;
e) Rotate the balloon by pulling the silicone sleeve while holding the endoscope static in position to expose the coupling element 120 of the first device attached to the balloon valve to be grasped by endoscopic forceps;
f) Use the forceps to grasp and pull the coupling element 120 to allow the balloon valve to be aligned and punctured using a catheter and going through the first device 100 until it passes the balloon valve mechanism to then inflate/deflate it; and
g) When finished inflating/deflating the balloon, the catheter is removed, and the valve is automatically closed.

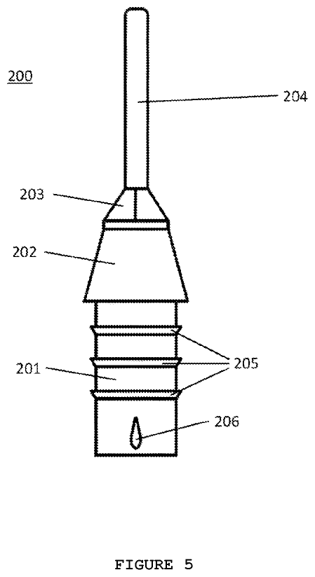

Still, according to a second realization of the present invention, exemplified by FIGS. 5, 6, 7, 8, 9 and 10, a second device 200 for bi-directional adjustment of an intragastric balloon is provided. The second device 200 is preferably manufactured of a biocompatible polymeric material.

As exemplified by FIG. 5, the second device 200 for bi-directional adjustment of an intragastric balloon comprises an upper body portion 201, an anchor portion 202, a restriction portion 203 and a tubular extension portion 204. The upper body portion 201 comprises at least three protrusions 205 and at least one projection 206.

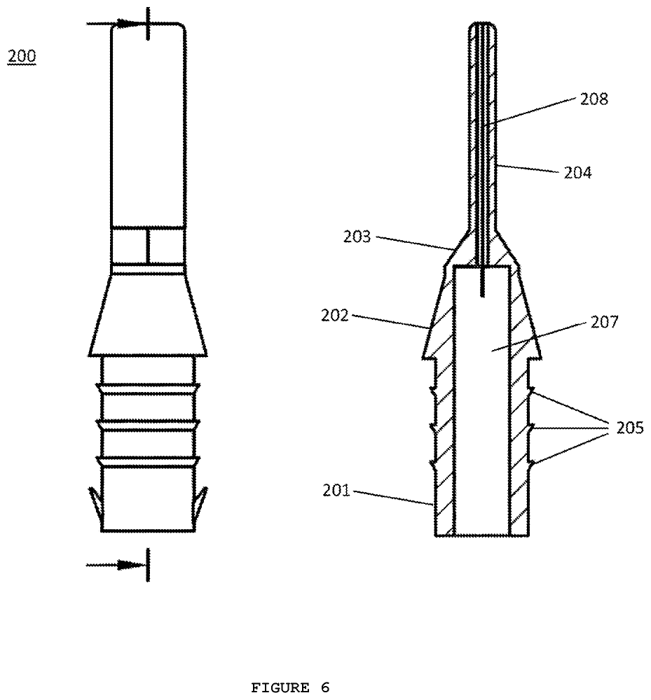

As exemplified by FIG. 6, the sectional view demonstrates that the second device further comprises a central cavity 207 and additionally a reduced central cavity 208 in the tubular extension portion 204.

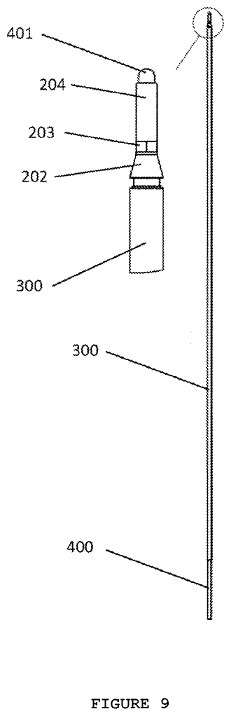

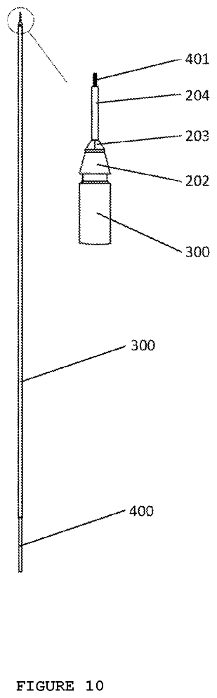

The upper body portion 201 has the function of providing a coupling with an implantable catheter 300, as exemplified by FIGS. 9 and 10. The implantable catheter 300 is preferably made of silicone.

The anchor portion 202 of the second device is configured to pass through the initial portion of the intragastric balloon valve to be retained in the valve.

The anchor portion 202 has a tapered shape, designed to pass through the initial portion of the intragastric balloon valve until the entire anchor portion 202 is inserted into the initial portion of the valve, causing the first device 200 to be retained in the intragastric balloon valve.

The restriction portion 203 is designed to support and provide rigidity to the tubular extension portion 204.

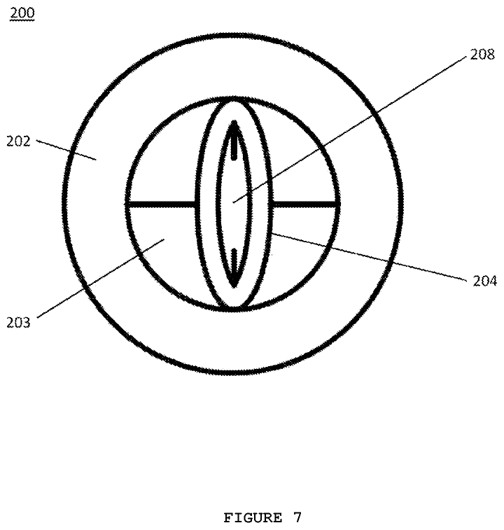

The tubular extension portion 204 is configured to pass through the valve of the intragastric balloon, thereby neutralizing its valve effect. Its elliptical shape, as exemplified by FIG. 7, is designed to create a seal with the inside of the balloon valve and prevent leakage from the side of the balloon valve. Further, the tubular extension portion 204 is designed to permit bi-directional adjustment by means of a reduced central cavity 208.

The tubular extension 204 may have an elliptical shape designed for better engagement with the distal portion of the balloon valve. However, its shape is not necessarily limited to an elliptical shape, it may have a substantially circular shape, a circular shape, and other shapes conducive to facilitate engagement with the distal portion of the balloon valve.

The at least three protrusions 205 and the at least one projection 206, as exemplified by FIGS. 5 and 6, are integrally formed to the second device 200 and configured to maintain the implantable catheter 300 coupled to the second device 200.

The second device 200 is coupled to the implantable catheter 300 by using a mandrel 400 preferably made of a metallic material, to give rigidity to the assembly, so that it can be introduced into the intragastric balloon valve, as exemplified by FIGS. 9 and 10. The mandrel further comprises a tip portion (401).

After insertion of the assembly into the intragastric balloon valve, the mandrel 400 is removed so that the balloon assembly, second device 200 and implantable catheter 300, are implanted in the patient.

Optionally, at the distal end of the implantable catheter 300 coupled to the second device 200, a shut off valve 800 may be attached. The shut off valve 800 has the function of allowing, in its open configuration, the inflation/deflation of fluid from the intragastric balloon and, in its closed configuration, preventing fluid leakage from the balloon.

The shut off valve 800 is made of a biocompatible polymer material. The shut off valve 800 comprises a threaded cap 810 and a fitting base 820.

The threaded cap 810 comprises a head 811 and a threaded stem 812. The head 811 further comprises a rounded end 811.1, a recess 811.2 and a base 811.3.

The recess 811.2 has the function of facilitating the handling of the shut off valve 800 when adjusting the intragastric balloon, such as to be to be easily grasped by a polypectomy strap.

The rounded end 811.1 of the head 811 of the shut off valve 800 has a round, oval, bulging or lentil shape, preferably round. The round shape reduces the incidence of ulcers/scratches in the gastric tract when adjusting for inflation/deflation of the intragastric balloon while in the patient's stomach.

The fitting base 820 comprises a top portion 821, a threaded portion 822 and a smooth portion 823. The fitting base 820 is hollowed out to allow fluid to pass through it. Wherein the threaded portion 822 comprises an internal thread 823.1 and the smooth portion comprises a central cavity 823.2.

The top portion 821 is preferably in contact with the base 811.3 of the head 811. Wherein the contact of the top portion 821 with the base 811.3 of the head 811 creates an airtight seal in conjunction with the screw tightness between the threaded cap 810 and the fitting base 820.

The smooth portion 823 of the fitting base 820 is for fixing an end of an implantable catheter 300 therein, preferably by means of a biocompatible glue. Optionally, the fitting base 820 of the shut off valve 800 may be manufactured integrally with the implantable catheter 300.

The present invention further comprises a method for performing adjustment of the intragastric balloon with the use of the second device 200 already in the stomach of the patient by means of an endoscope.

Said method comprises the following steps:
a) introduce an endoscopic tube orally into the patient until it reaches the stomach;
b) upon reaching the stomach, locate the balloon and the position of the 300 implantable catheter with the endoscope by visual means;
c) Grasp by endoscopic forceps the 300 implantable catheter;
d) With the endoscopic forceps, pull the 300 implantable catheter to stretch the catheter and lengthen it until it is externalized by the patient's oral route;
e) Inflate/deflate the balloon by means of the stretched and externalized 300 implantable catheter; and
f) Finish inflating/deflating the balloon and return the implantable 300 catheter orally to the patient's stomach, guiding it with the use of the endoscope.

Optionally, in steps c) and d), the implantable catheter 300 comprises a shut off valve 800 fixed at its distal end, whereby the shut off valve 800 with the implantable catheter 300 is seized, in its recess 811.2, by a polypectomy loop.

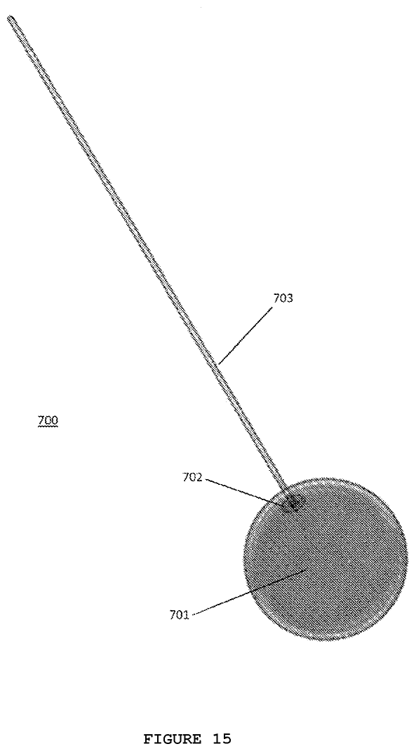

Further, in an embodiment of the present invention, an intragastric balloon assembly 700 containing an intragastric balloon 701 whose manufacture already includes a bidirectional adjustment device integral to the valve thereof is provided. This realization being favorable for reducing costs and the complexity of implementation of the other modalities of the present invention. Optionally, as illustrated in FIG. 15, the intragastric balloon may include a bi-directional adjustment device 702 and an implantable catheter 703 integral to the valve thereof.

Wherein the bi-directional adjustment device 702 is one of a first device 100 and a second device 200.

Those skilled in the art will value the knowledge presented herein and may reproduce the invention in the modalities presented and in other variants falling within the scope of the appended claims.

1. First device (100), characterized in that it is configured to be coupled to a valve of an intragastric balloon, said first device (100) comprises:
a body (110), wherein said body further comprises:
an upper body portion (111) comprising a lateral hole (114) and a lateral recess (115);
a neck portion (112);
an anchor portion (113);
a central cavity (116);
a coupling element (120); and
wherein the body (110) is configured to enter and be retained on the intragastric balloon valve;
wherein the coupling element (120) is configured to be coupled to the body (110) through the lateral hole (114) and protrude out of the intragastric balloon valve.

2. First device (100) according to claim 1, characterized in that the body (110) is completely introduced into the intragastric balloon valve to be retained therein by means of the anchor portion (113).

3. First device (100) according to claim 2, further characterized in that the anchor portion (113) is tapered.

4. First device (100) according to claim 1, characterized in that the neck portion (112) is configured to provide a fluid seal when in contact with the walls of the intragastric balloon valve.

5. First device (100) according to claim 1, characterized by the fact that the lateral hole (114) goes through the side of the first device (100).

6. First device (100) according to claim 1, characterized in that the lateral groove (115) is positioned adjacent the lateral hole (114), and is configured to allow the coupling element (120) to be removably engaged therein when the coupling element (120) is pulled.

7. First device (100) according to claim 1, further characterized in that it is configured to be coupled to a device (500) for inflation or deflation of the intragastric balloon.

8. First device (100) according to claim 1, characterized in that the body (110) of the first device (100) is preferably manufactured from a biocompatible polymeric material.

9. First device (100) according to claim 1, characterized in that a mandrel (400) is used for introducing the body (110) into the intragastric balloon valve.

10. First device (100) according to claim 9, characterized in that the mandrel (400) is preferably manufactured from a metallic material.

11. First device (100) according to claim 9, characterized in that the mandrel (400) is inserted into the central chamber (116) of the body (110), for inserting the device into the intragastric balloon valve.

12. First device (100) according to claim 9, characterized in that, after insertion of the first device (100) into the intragastric balloon valve, the mandrel (400) is removed from the first device (100).

13. Method of adjusting intragastric balloons comprising the first device as defined by claim 1, said method characterized in that it comprises the following steps:
a) place a silicone sleeve over the endoscopy tube before insertion into the patient;
b) introduce the endoscopy tube with the silicone sleeve orally into the patient until it reaches the stomach;
c) when the stomach is reached, the balloon is located with the endoscope by visual means;
d) after visually identifying that the balloon valve is in a position that prevents access to the coupling element (120), pass the endoscope through the side of the balloon;
e) rotating the balloon by pulling the silicone sleeve, keeping the endoscope static in its position, whereby exposing the coupling element (120) of the first device attached to the balloon valve, which is then grasped by endoscopic forceps;
f) use the forceps to grasp and pull the coupling element (120) allowing it to be aligned and the balloon valve to be punctured using a catheter, passing it through the first device (100) until it passes the balloon valve mechanism to inflate or deflate the balloon; and
g) after finishing inflating or deflating the balloon, the catheter is removed and the valve closes automatically.

14. Second device (200), characterized in that it is configured to be coupled to a valve of an intragastric balloon, said second device (200) comprises:
an upper body portion (201);
an anchor portion (202);
a restriction portion (203);
a tubular extension portion (204), said tubular extension portion (204) further comprising a reduced central cavity (208);
at least one protrusion (205); at least one projection
a central cavity (207); and
wherein the body portions (202, 203) are configured to be introduced into, and retained by, the intragastric balloon valve.

15. Second device (200) according to claim 14, characterized in that the upper body portion (201) is configured to be coupled with an implantable catheter (300).

16. Second device (200) according to claim 15, characterized in that the implantable catheter (300) is preferably manufactured from silicone.

17. Second device (200) according to claim 16, characterized in that the upper body portion (201) further comprises at least three protrusions (205) and at least one projection (206).

18. Second device (200) according to claim 17, characterized in that the at least three protrusions (205) are configured to hold the implantable catheter (300) coupled to the second device (200).

19. Second device (200) according to claim 17, characterized in that the at least one projection (206) is configured to hold the implantable catheter (300) coupled to the second device (200).

20. Second device (200) according to claim 14, characterized in that the tubular extension portion (204) has a circular, and preferably elliptical shape.

21. Second device (200) according to claim 14, characterized in that the tubular extension portion (204) creates a seal with the inner part of the balloon valve and prevents leakage from the side thereof.

22. Second device (200) according to claim 14, characterized in that the tubular extension portion (204) is further designed so that the bi-directional adjustment of the intragastric balloon is by means of a reduced central cavity (208).

23. Second device (200) according to claim 14, characterized in that the anchor portion (202) is further configured to retain the second device in the intragastric balloon valve.

24. Second device (200) according to claim 23, characterized in that the anchor portion (202) has a tapered shape.

25. Second device (200) according to claim 14, characterized in that restriction portion (203) is designed to support and provide rigidity to the tubular extension portion (204).

26. Second device (200) according to claim 14, characterized in that the tubular extension portion (204) is configured to pass through the intragastric balloon valve, thereby neutralizing its valve effect.

27. Second device (200) according to claim 14, characterized in that the body (110) of the first device (100) is preferably made of a biocompatible polymeric material.

28. Shut off valve (800), characterized in that it is configured to be coupled to an implantable catheter (300) of an intragastric balloon setting means, said shut off valve (800) comprising:
a threaded cap (810), wherein said threaded cap (810) further comprises:
a head (811) and a threaded stem (812), wherein said head comprises a rounded end (811.1), a recess (811.2) and a base (811.3);
a fitting base (820), wherein said threaded cap (820) further comprises:
a top part (821), a threaded part (822) and a smooth part (823).

29. Shut off valve (800) according to claim 28, characterized in that it has an open and a closed configuration.

30. Shut off valve (800) according to claim 29, characterized in that in the open configuration, the threaded cap (810) is off the fitting base (820), and in the closed configuration the threaded cap (810) is threaded into the fitting base (820).

31. Shut off valve (800) according to claim 28, characterized in that it is manufactured from a biocompatible polymeric material.

32. Shut off valve (800) according to claim 28, characterized in that the recess (811.2) allows handling of the shut off valve (800) when seized by a polypectomy strap.

33. Shut off valve (800) according to claim 28, characterized in that the rounded end 811.1 of the head has a round, oval, bulging or lentil shape, preferably a round shape.

34. Shut off valve (800) according to claim 28, characterized in that the threaded portion (822) and the smooth portion (823) comprise, respectively, an internal thread (823.1) and a central cavity (823.2).

35. Shut off valve (800) according to claim 28, characterized in that the fitting base (820) is fixed to an implantable catheter (300) by means of a biocompatible glue.

36. Shut off valve (800) according to claim 28, characterized in that the fitting base (820) is manufactured integrally with an implantable catheter (300).

37. Shut off valve (800) according to claim 28, characterized in that it is used in conjunction with an implantable catheter (300) coupled to a second device (200) for bi-directional adjustment of an intragastric balloon.

38. Intragastric balloon assembly (700), characterized for comprising:
an intragastric balloon (701);
a bi-directional adjustment device (702); and
optionally, an implantable catheter (703);
wherein the bi-directional adjustment device (702) is integral to the balloon valve (703).
where the implantable catheter (703) is integral to the intragastric balloon valve.

39. Assembly (700) according to claim 38, characterized in that the bi-directional adjustment device (702) is one of a first device (100) and a second device (200).
